(19) United States
(12) Patent Application Publication
(10) Pub. No.: US 2009/0177182 A1
(43) Pub. Date: Jul. 9, 2009

(54) Glass Drug Chamber For Automated Ophthalmic Injection Device

(76) Inventors: Dyson W. Hickingbotham, Stouchsberg, PA (US); Robert J. Sanchez, JR., Oceanside, CA (US)

Correspondence Address: ALCON, IP LEGAL, TB4-8, 6201 SOUTH FREEWAY, FORT WORTH, TX, 76134, US

(21) Appl. No.: 11/971,443

(22) Filed: Jan. 9, 2008

(51) Int. Cl. A61F 9/00; A61M 5/44

(52) U.S. Cl. 604/506; 604/113

(57) ABSTRACT

A dispensing assembly has a glass tube assembly, a chamber housing, a needle, and a temperature control device. The glass tube assembly includes a glass tube with inner and outer surfaces and distal and proximal ends. A plunger is fluidly sealed to the inner surface of the glass tube at the proximal end, and a seal is located at the distal end of the glass tube. The chamber housing has an inner surface and an outer surface. The inner surface partially defines a chamber for receiving the glass tube assembly. The needle has sharp distal and proximal ends, traverses the chamber housing, and is fluidly coupled to the chamber. The temperature control device at least partially surrounds the chamber housing. The proximal end of the needle is arranged to puncture the seal when the glass tube assembly is pressed against the proximal end of the needle.

### BACKGROUND OF THE INVENTION

The present invention relates to a single-use medical device and more particularly to a two-piece ophthalmic drug delivery device with a disposable tip end that uses a glass drug chamber.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually performed using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to pierce the eye tissue with the needle, hold the syringe steady, and actuate the syringe plunger (with or without the help of a nurse) to inject the fluid into the eye. The volume injected is typically not controlled in an accurate manner because reading the vernier is subject to parallax error. Fluid flow rates are uncontrolled. and tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for injecting a drug into the eye. Such a hand piece can include a limited reuse assembly attachable to and removable from a disposable tip segment. Properly dosing and delivering the drug can be challenging. A pre-set dosage of a drug suspended in a phase transition material must be heated before being delivered into the eye. This requires placing the material in a drug chamber that can be heated. Properly containing the material in a glass drug chamber would be advantageous.

### SUMMARY OF THE INVENTION

In one embodiment consistent with the principles of the present invention, the present invention is a dispensing assembly having a glass tube assembly, a chamber housing, a needle, and a temperature control device. The glass tube assembly includes a glass tube with inner and outer surfaces and distal and proximal ends. A plunger is fluidly sealed to the inner surface of the glass tube at the proximal end, and a seal is located at the distal end of the glass tube. The chamber housing has an inner surface and an outer surface. The inner surface partially defines a chamber for receiving the glass tube assembly. The needle has sharp distal and proximal ends, traverses the chamber housing, and is fluidly coupled to the chamber. The temperature control device at least partially surrounds the chamber housing. The proximal end of the needle is arranged to puncture the seal when the glass tube assembly is pressed against the proximal end of the needle.

In another embodiment consistent with the principles of the present invention, the present invention is a dispensing assembly having a glass tube assembly, a chamber housing, a needle, and a heater. The glass tube assembly includes a glass tube with inner and outer surfaces and distal and proximal ends. A plunger is fluidly sealed to the inner surface of the glass tube at the proximal end, and a seal is located at the distal end of the glass tube. The glass tube contains a drug suspended in a phase transition compound. The chamber housing has an inner surface and an outer surface. The inner surface partially defines a chamber for receiving the glass tube assembly. The needle has sharp distal and proximal ends, traverses the chamber housing, and is fluidly coupled to the chamber. The heater at least partially surrounds the chamber housing and heats the drug and phase transition compound. The proximal end of the needle is arranged to puncture the seal when the glass tube assembly is pressed against the proximal end of the needle.

In another embodiment consistent with the principles of the present invention, the present invention is a method of injecting a drug suspended in a phase transition compound into an eye. The method includes: providing a glass tube assembly comprising a glass tube having inner and outer surfaces and distal and proximal ends, a plunger fluidly sealed to the inner surface of the glass tube at the proximal end, and a seal located at the distal end of the glass tube, the glass tube containing a drug suspended in a phase transition compound; heating the glass tube assembly after it is located in a chamber housing having an inner surface and an outer surface, the inner surface partially defining a chamber for receiving the glass tube assembly; puncturing the seal with a needle having sharp distal and proximal ends, the needle located in the chamber housing and fluidly coupled to the chamber; and dispensing the drug suspended in the phase transition compound by moving the plunger toward the needle.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying figures, which are incorporated in and constitute a part of this specification, illustrate several embodiments of the invention and together with the description, serve to explain the principles of the invention.

FIG. 1 is a perspective view of a prior art syringe.

FIG. 2 is a view of an ophthalmic medical device including a disposable tip segment and a limited reuse assembly according to the principles of the present invention.

FIG. 3 is an embodiment of a limited reuse assembly according to the principles of the present invention.

FIG. 4 is a cross section view of a disposable tip segment and a limited reuse assembly according to the principles of the present invention.

FIG. 5 is a cross section view of a disposable tip segment according to the principles of the present invention.

FIG. 6 is a cross section view of a chamber housing assembly according to the principles of the present invention.

FIG. 7 is a cross section view of a glass tube assembly according to the principles of the present invention.

FIGS. 8A & 8B are cross section views of a glass tube assembly and a chamber housing assembly according to the principles of the present invention.

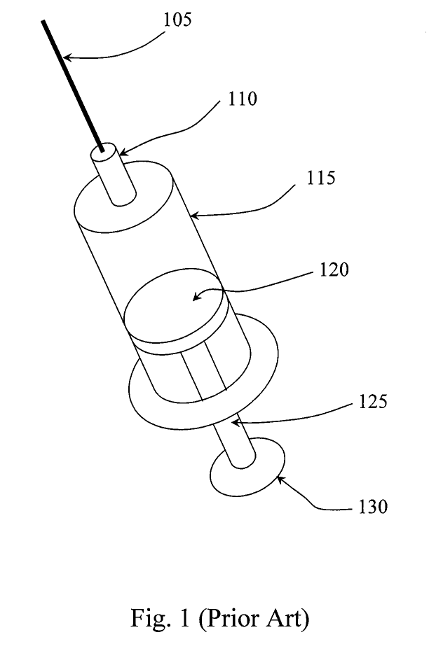

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

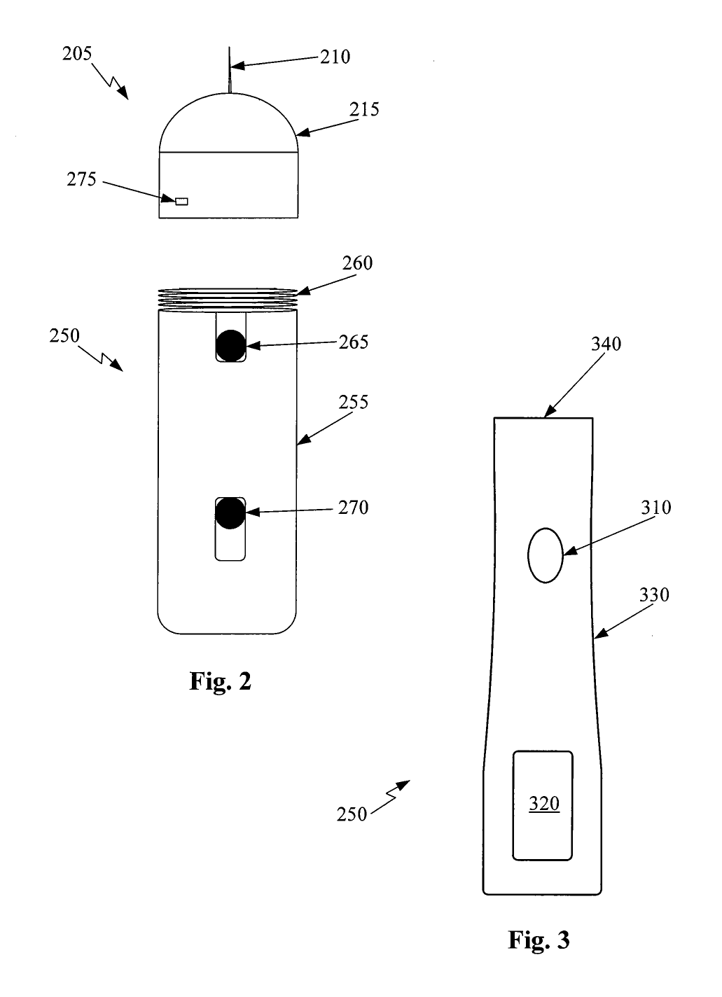

FIG. 2 depicts one view of an ophthalmic medical device including a disposable tip segment and a limited reuse assembly according to an embodiment of the present invention. In FIG. 2, the medical device includes a tip segment 205 and a limited reuse assembly 250. The tip segment 205 includes a needle 210, a housing 215, and an optional light 275. The limited reuse assembly 250 includes a housing 255, a switch 270, a lock mechanism 265, and a threaded portion 260.

Tip segment 205 is capable of being connected to and removed from limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug based on thermal characteristics.

Switch 270 is adapted to provide an input to the system. For example, switch 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of disposable tip segment 205. For example, optional light 275 may also pulse on and off to indicate other states, such as, but not limited to a system error, fully charged battery, insufficiently charged battery or faulty connection between the tip segment 205 and limited use assembly 250. While shown on tip segment 205, optional light 275 or other indicator may be located on limited reuse assembly 250.

FIG. 3 is another embodiment of a limited reuse assembly according to the principles of the present invention. Limited reuse assembly 250 includes a button 310, a display 320, and a housing 330. Disposable tip segment 205 attaches to end 340 of limited reuse assembly 250. Button 310 is actuated to provide an input to the system. As with switch 270, button 310 may activate a heater or other temperature control device or initiate actuation of a plunger. Display 320 is a liquid crystal display, segmented display, or other device that indicates a status or condition of disposable tip segment 205 or limited reuse assembly 250.

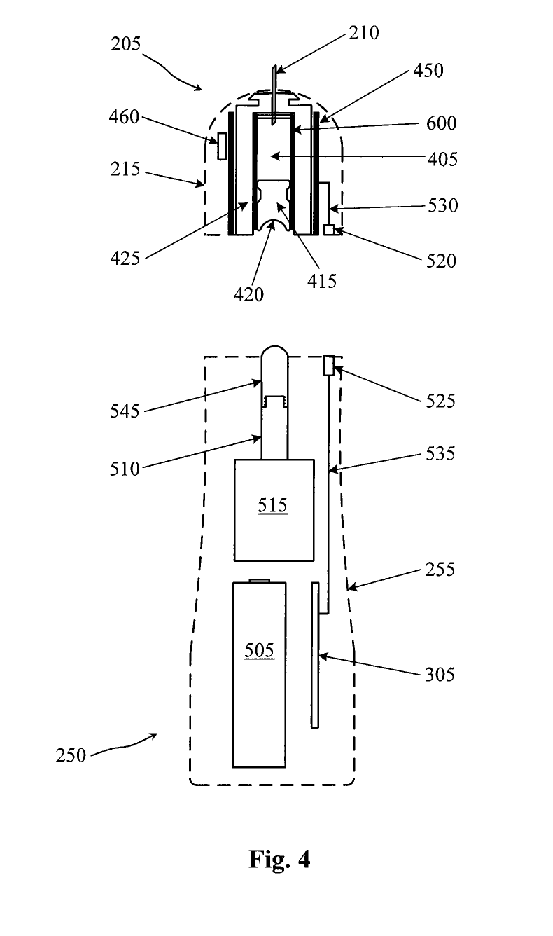

FIG. 4 is a cross section view of a disposable tip segment and a limited reuse assembly according to an embodiment of the present invention. FIG. 4 shows how tip segment 205 interfaces with limited reuse assembly 250. In the embodiment of FIG. 4, tip segment 205 includes plunger interface 420, plunger 415, chamber housing 425, tip segment housing 215, temperature control device 450, thermal sensor 460, needle 210, chamber 405, interface 530, and tip interface connector 520. Limited reuse assembly 250 includes mechanical linkage 545, actuator shaft 510, actuator 515, power source 505, controller 305, limited reuse assembly housing 255, interface 535, and limited reuse assembly interface connector 525.

Needle 210 is fluidly coupled to chamber 405. In such a case, a substance contained in a glass tube assembly 600 in chamber 405 can pass through needle 210 and into an eye. Temperature control device 450 at least partially surrounds chamber housing 425. In this case, temperature control device 450 is adapted to heat and/or cool chamber housing 425 and any substance contained in chamber 405. Interface 530 connects temperature control device 450 with tip interface connector 520.

In limited reuse assembly 250, power source 505 provides power to actuator 515. An interface (not shown) between power source 505 and actuator 515 serves as a conduit for providing power to actuator 515. Actuator 515 is connected to actuator shaft 5 10. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage interface 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and plunger interface 420 respectively.

Controller 305 and actuator 515 are connected by an interface (not shown). This interface (not shown) allows controller 305 to control the operation of actuator 515. In addition, an interface (not shown) between power source 505 and controller 305 allows controller 305 to control operation of power source of 310. In such a case, controller 305 may control the charging and the discharging of power source 505 when power source 505 is a rechargeable battery.

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component, controller 305 may be made of many different components or integrated circuits.

Tip segment 205 is adapted to mate with or attach to limited reuse assembly 250 as previously described. In the embodiment of FIG. 4, plunger interface 420 located on a bottom surface of plunger 415 is adapted to mate with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 525, tip interface connector 520, and interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is mated with plunger interface 420, moves plunger 415 upward toward needle 210. A substance located in a glass tube assembly 600 in chamber 405 is then expelled through needle 210.

In addition, controller 305 controls the operation of temperature control device 450. Temperature control device 450 is adapted to heat and/or cool chamber housing 425. Since chamber housing 425 is at least partially thermally conductive, heating or cooling chamber housing 425 heats or cools a substance located in a glass tube assembly 600 in chamber 405. Temperature information can be transferred from thermal sensor 460 to controller 305 via any of a number of different interface configurations. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

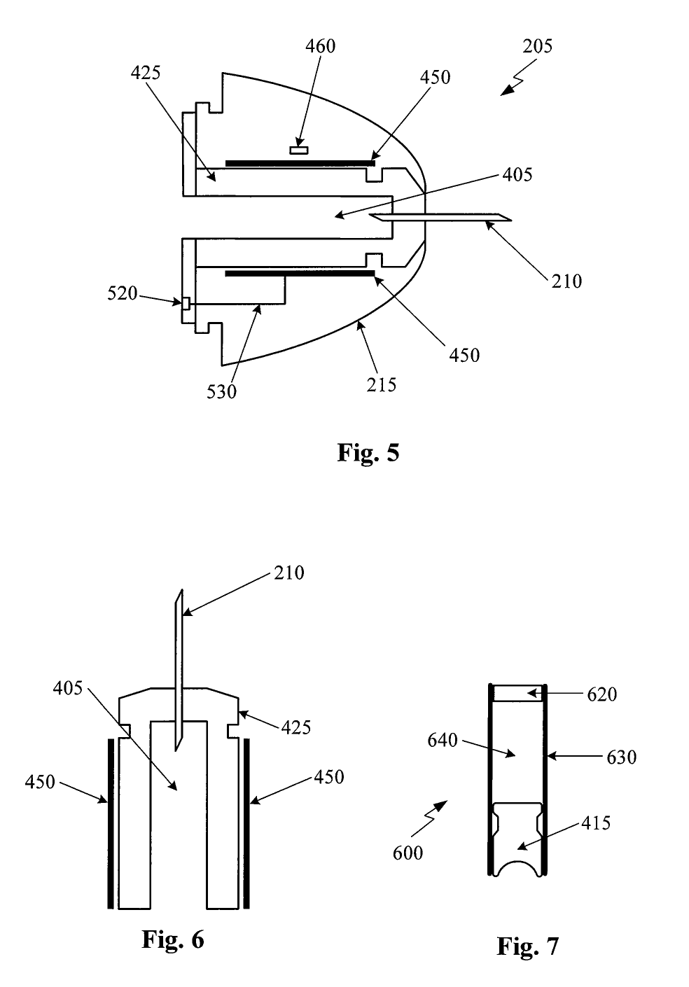

FIG. 5 is a cross section view of a disposable tip segment for an ophthalmic medical device according to an embodiment of the present invention. In FIG. 5, disposable tip segment 205 includes housing 215, needle 210, chamber 405, chamber housing 425, temperature control device 450, thermal sensor 460, interface 530, and tip interface connector 520. Disposable tip segment 205 operates as a disposable injection device.

In the embodiment of FIG. 5, needle 210 is fluidly coupled to chamber 405. Temperature control device 450 at least partially surrounds chamber housing 425 and chamber 405. Housing 215 forms an outer skin on disposable tip segment 205.

In various embodiments of the present invention, temperature control device 450 is a heating and/or a cooling device. Temperature control device 450 is in thermal contact with chamber housing 425. As such, temperature control device 450 is capable of changing the temperature of the substance (in a glass tube assembly) in chamber 405. Interface 530 and tip interface connector 520 couple temperature control device 450 to a limited reuse assembly. In such a case, temperature control device 450 can be powered and controlled by the limited reuse assembly.

A substance to be delivered into an eye, typically a drug, is located in a glass tube assembly in chamber 405. Typically, chamber 405 is cylindrical in shape. Temperature control device 450 is in thermal contact with chamber housing 425. In this manner, temperature control device 450 is adapted to control the temperature of the contents of chamber 405. Thermal sensor 460 provides temperature information to assist in controlling the operation of temperature control device 450.

In one embodiment of the present invention, the substance located in chamber 405 is a drug that is preloaded into sealed glass tube assembly 600 as depicted in FIG. 7. When a drug is preloaded into chamber 405, a set quantity of the drug can be preloaded. For example, 100 microliters of a drug can be loaded into chamber 405, and any quantity up to 100 microliters can be dispensed. In such a case, the plunger 415 can be moved a precise distance to deliver a precise dosage of drug from the chamber 405, through the needle 210, and into an eye. This provides for flexibility of dosing and for ease of assembly.

FIG. 6 is a cross section view of a chamber housing assembly according to the principles of the present invention. In FIG. 6, temperature control device 450 at least partially surrounds chamber housing 425. Chamber 405 is bounded by an interior surface of chamber housing 425. Needle 210 has two sharp ends—one for piercing the eye and the other in chamber 405. The glass tube assembly 600 of FIG. 7 fits in chamber 405.

FIG. 7 is a cross section view of a glass tube assembly 600 according to the principles of the present invention. Glass tube 630 has a seal 620 on one end and a plunger 415 on the other. In this manner, a sealed drug chamber 640 is bounded by these components. This sealed chamber provides a safe environment for storing and transporting a drug.

Glass tube 630 is a tube made of glass. As is commonly known, glass is a suitable material for contact with pharmaceuticals. A drug suspended in a phase transition compound is contained in glass tube 630. The drug/compound mixture can be heated, since glass conducts heat, and can be carried in the glass tube until it is injected into an eye.

Seal 620 and plunger 415 are located on either end of glass tube 630 as shown. Both seal 620 and plunger 415 are sealed to an interior surface of glass tube 630. Seal 620 is preferably a self sealing elastomer that can be in the shape of a plug or disc. The outer surface of plunger 415 forms a fluidic seal with the inner surface of glass tube 630. In this manner, drug chamber 640 is a sealed chamber that contains a drug or other substance that can be injected into an eye. Drug chamber 640 is a sealed chamber that allows the proper distribution and handling of a drug dosage. For example, a number of different glass tube assemblies can be manufactured—each with different drug dosages. A doctor can select the proper dosage and insert the glass tube assembly 600 into chamber 405 for injection into the eye.

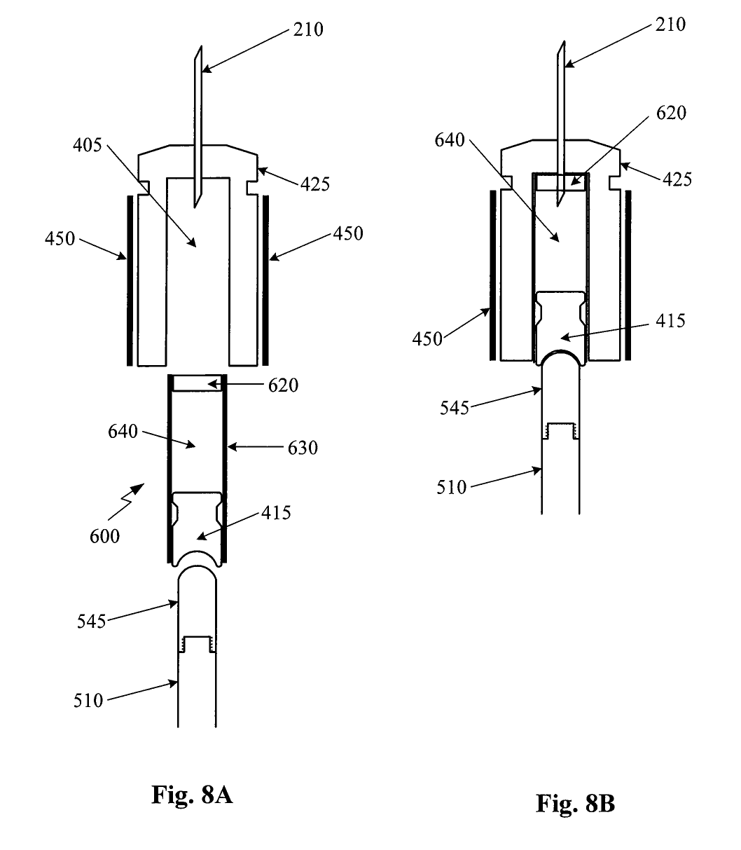

FIGS. 8A and 8B are cross section views of a glass tube assembly 600 and a chamber housing assembly according to the principles of the present invention. FIGS. 8A and 8B show how the glass tube assembly 600 is placed in chamber 405. The glass tube assembly 600 is inserted into chamber 405 such that the exterior surface of glass tube 630 at least partially contacts the interior surface of chamber housing 425. Mechanical linkage interface 545 (driven by actuator shaft 510) can be used to push the glass tube assembly into chamber 405 and to pierce seal 620 (as shown in FIG. 8B). The sharp end of needle 210 located in chamber 405 pierces seal 620.

In one embodiment, a user inserts a glass tube assembly 600 into chamber 405 by hand, much like loading a shotgun shell into a shotgun. The disposable tip segment 205 that includes the chamber 405 can then be placed on a limited reuse assembly 250. The temperature control device 450 is activated to heat the substance contained in drug chamber 640 when the substance is a drug suspended in a phase transition compound. The plunger is then moved forward to dispense the substance into the eye.

In one case, the glass tube assembly is heated before it is pushed forward against the needle to pierce seal 620. In this manner, the drug is properly heated before the seal 620 is pierced to deliver the drug. The mechanical linkage interface 545 can operate in two phases. In the first phase, the mechanical linkage interface 545 pushes the glass tube assembly 600 forward so that it can be heated. In the second phase, the mechanical linkage interface pushes the glass tube assembly 600 forward so that the seal 620 is pierced, and the contents of drug chamber 640 are delivered into the eye.

From the above, it may be appreciated that the present invention provides an improved system for delivering precise volumes of a substance into an eye. The present invention provides a single use, disposable delivery device tip segment that is capable of delivering a precise dosage. The tip segment interfaces with a limited reuse assembly. The disposable tip segment has a chamber that receives a glass tube assembly containing a drug. The glass tube assembly is loaded into the disposable tip segment and the injection process is activated. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A dispensing assembly comprising:
a glass tube assembly comprising a glass tube having inner and outer surfaces and distal and proximal ends, a plunger fluidly sealed to the inner surface of the glass tube at the proximal end, and a seal located at the distal end of the glass tube;
a chamber housing having an inner surface and an outer surface, the inner surface partially defining a chamber for receiving the glass tube assembly;
a needle having sharp distal and proximal ends, the needle traversing the chamber housing and fluidly coupled to the chamber;
a temperature control device at least partially surrounding the chamber housing;
wherein the proximal end of the needle is arranged to puncture the seal when the glass tube assembly is pressed against the proximal end of the needle.

2. The assembly of claim 1 wherein the seal is made of a self-sealing elastomer.

3. The assembly of claim 1 wherein the glass tube contains a drug suspended in a phase transition compound.

4. The assembly of claim 4 wherein the temperature control device is a heater for heating the drug suspended in the phase transition compound.

5. The assembly of claim 1 further comprising:
a housing at least partially enclosing the chamber housing.

6. The assembly of claim 1 further comprising:
a thermal sensor in thermal contact with the chamber housing.

7. The assembly of claim 1 further comprising:
an actuator having a shaft; and
a mechanical linkage interface for engaging the plunger.

8. The assembly of claim 7 wherein the mechanical linkage interface is arranged to contact the plunger and press the seal against the proximal end of the needle to puncture the seal.

9. A dispensing assembly comprising:
a glass tube assembly comprising a glass tube having inner and outer surfaces and distal and proximal ends, a plunger fluidly sealed to the inner surface of the glass tube at the proximal end, and a seal located at the distal end of the glass tube, the glass tube containing a drug suspended in a phase transition compound;
a chamber housing having an inner surface and an outer surface, the inner surface partially defining a chamber for receiving the glass tube assembly;
a needle having sharp distal and proximal ends, the needle traversing the chamber housing and fluidly coupled to the chamber;
a heater at least partially surrounding the chamber housing, the heater for heating the drug and phase transition compound;
wherein the proximal end of the needle is arranged to puncture the seal when the glass tube assembly is pressed against the proximal end of the needle.

10. The assembly of claim 1 wherein the seal is made of a self-sealing elastomer.

11. The assembly of claim 1 further comprising:
a housing at least partially enclosing the chamber housing.

12. The assembly of claim 1 further comprising:
a thermal sensor in thermal contact with the chamber housing.

13. A method of injecting a drug suspended in a phase transition compound into an eye, the method comprising:
providing a glass tube assembly comprising a glass tube having inner and outer surfaces and distal and proximal ends, a plunger fluidly sealed to the inner surface of the glass tube at the proximal end, and a seal located at the distal end of the glass tube, the glass tube containing a drug suspended in a phase transition compound;
heating the glass tube assembly after it is located in a chamber housing having an inner surface and an outer surface, the inner surface partially defining a chamber for receiving the glass tube assembly;
puncturing the seal with a needle having sharp distal and proximal ends, the needle located in the chamber housing and fluidly coupled to the chamber; and
dispensing the drug suspended in the phase transition compound by moving the plunger toward the needle.

14. The method of claim 13 further comprising:
providing an interface between a tip segment containing the chamber housing and a limited reuse assembly.

15. The method of claim 13 wherein puncturing the seal with a needle having sharp distal and proximal ends further comprises providing force on the plunger to drive the seal into the proximal end of the needle.

16. The method of claim 13 wherein puncturing the seal with a needle having sharp distal and proximal ends further comprises providing force on the plunger to drive the seal into the proximal end of the needle after the drug suspended in the phase transition compound has been heated.
